(19) United States
(12) Patent Application Publication
(10) Pub. No.: US 2017/0172585 A1
(43) Pub. Date: Jun. 22, 2017

(54) SYSTEM, APPARATUS, AND METHOD FOR CREATION AND IMPLANTATION OF TISSUE GRAFTS

(71) Applicant: THE CURATORS OF THE UNIVERSITY OF MISSOURI, Columbia, MO (US)

(72) Inventors: Ferris M. Pfeiffer, Columbia, MO (US); James L. Cook, Columbia, MO (US); James P. Stannard, Columbia, MO (US)

(73) Assignee: The Curators of the University of Missouri, Columbia, MO (US)

(21) Appl. No.: 15/129,570

(22) PCT Filed: Mar. 30, 2015
(86) PCT No.: PCT/US15/23307

§ 371 Date: Sep. 27, 2016

Related U.S. Application Data
(60) Provisional application No. 61/973,036, filed on Mar. 31, 2014.

(51) Int. Cl. A61B 17/16; A61B 17/15; A61B 17/17

(52) U.S. Cl. CPC A61B 17/1635; A61B 17/17; A61B 17/15; A61B 17/1615

(57) ABSTRACT

Systems, apparatus, and methods for forming a non-circular tissue graft from donor tissue, and a corresponding recipient site in patient's tissue, are disclosed. In one embodiment, a cutting guide is disclosed for use in forming the recipient site. The cutting guide comprises a deformable body including a non-circular window, through which the patient's tissue is accessed, that is reconfigurable to contour to the patient's tissue. A cutting bit is also disclosed for use with the cutting guide in forming the recipient site. The cutting bit engages and follow a cutting route defined by the cutting guide, and includes a shaft, as well as a cutting head that removes portions of the patient's tissue. A cutting guide for use in forming the tissue graft from the donor tissue is also disclosed, which includes a removable template defining a channel that corresponds in configuration and dimensions to the cutting route.

### RELATED APPLICATION

This application claims the benefit of, and priority to, U.S. Provisional Patent Application Ser. No. 61/973,036, entitled “System, Apparatus, and Method for Creation and Implantation of Tissue Grafts,” filed Mar. 31, 2014, the entire content of which is hereby incorporated by reference.

### BACKGROUND

1. Field

The present disclosure relates generally to the grafting of tissue. More specifically, the present disclosure describes systems, apparatus, and methods useful in the creation of tissue grafts, e.g., osteochondral allografts, autografts, etc., and corresponding recipient sites in tissue that are configured and dimensioned to receive the tissue grafts. In certain embodiments, the systems, apparatus, and methods described herein create tissue grafts and corresponding recipient sites that are non-circular in configuration.

2. Discussion

Common usages for tissue grafts may include the treatment of cartilage defects to restore normal joint function. For example, an osteochondral allograft (OCA) is a type of tissue graft commonly used to treat cartilage defects resulting from osteochondrosis, trauma, and osteoarthritis, as well as osteochondritis dessicans (OCD) and focal lesions of osteoarthritis, which encompass a significant and increasing number of patients (estimated 600,000-900,000 per year in the United States).

Known OCA techniques sometimes utilize allografts harvested from donor tissue that are generally circular in shape, which are inserted into a similarly configured recipient site, e.g., a cavity, socket, or the like. Such grafts, however, while relatively easy to create and transplant, are not optimal for use in repairing certain defects, e.g., defects with large aspect ratios, and/or defects that are non-circular in configuration.

Currently, non-circular defects are often repaired using multiple OCAs that are circular in configuration, a procedure often referred to as the “snowman” technique as it consists of overlapping circular grafts in an attempt to best cover the defect. In addition to being technically difficult to perform, this technique has a number of drawbacks that often reduce the success and/or benefits of the procedure including, for example, the inefficient use of donor tissue, non-anatomical reconstruction, the removal of significant amounts of healthy tissue to accommodate for the circular configuration of the grafts, and compromised graft stability.

Accordingly, a need remains for systems, apparatus, and methods that provide the clinical advantages of a known grafting procedures, but overcome the limitations of current instrumentation and methodologies.

### SUMMARY

The systems, apparatus, and methods described herein are adapted to form a tissue graft, as well as a corresponding recipient site in tissue that is configured and dimensioned to receive the tissue graft.

In one aspect of the present disclosure, the formation of a non-circular tissue graft is described. In certain embodiments, the non-circular tissue graft comprises a distal (application) side and a proximal (exposed) side separated by a distance equal to the thickness of the tissue graft. During placement, the non-circular tissue graft is oriented such that the distal side faces the recipient site.

In certain embodiments, the non-circular tissue graft may define a length (L) and a width (W), wherein the length (L) may be greater than the width (W) such that the aspect ratio (L:W), i.e., the ratio of the length (L) to the width (W), may be greater than 1.0, e.g., 1.5 or greater.

In another aspect of the present disclosure, a recipient site cutting guide is described that comprises a working surface, a mounting surface extending in substantially coplanar relation to the working surface, a guide wall defining a height that is positioned between the working surface and the mounting surface such that the working surface is separated from the mounting surface by a distance equal to the height of the guide wall, and a cutting route that is positioned in a plane extending in substantially perpendicular relation to the working surface.

In certain embodiments, the recipient site cutting guide may include, e.g., be formed from, a material that is selected from the group consisting of plastic, aluminum, stainless steel, titanium, and combinations thereof.

In certain embodiments, the working surface and the cutting route of the recipient site cutting guide may be adapted to engage a cutting bit that is configured and dimensioned to remove tissue.

In certain embodiments, the cutting route may be defined by at least a portion of the guide wall.

In certain embodiments, the cutting route may be non-circular in configuration, e.g., the cutting route may be oval, ovoid, triangular, rectangular, or combinations thereof.

In certain embodiments, the cutting route may be discontinuous, defining discrete endpoints.

In certain embodiments, the cutting route may define a cutting area having a length and a width. The length (L) may be greater than the width (W) such that the aspect ratio (L:W), i.e., the ration of the length (L) to the width (W), is greater than 1.0.

In certain embodiments, the recipient site cutting guide may further comprise a mounting portion that is adapted to couple the recipient site cutting guide to a recipient site in a patient's tissue. For example, the recipient site cutting guide may include at least one mounting eyelet that extends through the working surface and the mounting surface for receipt of a mounting pin, screw, or other such structure.

In certain embodiments, the recipient site cutting guide may comprises a plurality of reliefs formed in the mounting surface, which may extend into the guide wall.

The mounting surface may define a plurality of contact points, at least one of which is separated from the remainder of the mounting surface by at least one of the reliefs.

In certain embodiments, the contact points may be moveable relative to one another via manipulation, e.g., reconfiguration, of the recipient site cutting guide. To facilitate such reconfiguration, the guide wall may be deformable, e.g., plastically deformable.

In another aspect of the present disclosure, a grafting system is described for forming, e.g., cutting, a tissue graft. The grafting system includes a recipient site cutting guide comprising a working surface, a mounting surface that extends in substantially coplanar relation to the working surface, a guide wall defining a height that is positioned between the working surface and the mounting surface such that the working surface is separated from the mounting surface by a distance equal to the height of the guide wall, and a cutting route that is positioned in a plane extending in substantially perpendicular relation to the working surface.

The grafting system may further comprise a cutting bit configured and dimensioned to remove tissue that comprises an elongate shaft having a length defined between opposite (first and second) ends, an arbor that is positioned at the first end of the elongate shaft, a cutting head that is positioned at the second end of the elongate shaft, and a depth guide that is positioned on the elongate shaft between the first and second ends.

In certain embodiments, the depth guide may include an engaging surface defining a distally facing end wall that is configured, dimensioned, and adapted to engage the working surface of the recipient site cutting guide.

In certain embodiments, the depth guide may be moveable along the length of the elongate shaft. For example, the depth guide may be releasably secured to the elongate shaft.

In certain embodiments, the depth guide may further comprise a collar that is adapted to be positioned about an external surface of the elongate shaft.

In certain embodiments, the depth guide may define an external diameter larger than an external diameter defined by the elongate shaft.

In another aspect of the present disclosure, a method for cutting a recipient site in a patient's tissue is disclosed that is configured an dimensioned to receive a corresponding tissue graft. The method includes providing a recipient site cutting guide defining a cutting route, deforming the recipient site cutting guide such that the recipient site cutting guide is contoured to the patient's tissue, securing the recipient site cutting guide to the patient's tissue such that the cutting route surrounds at least a portion of the area that will define the recipient site, providing a cutting bit with a depth guide and a cutting head, wherein the cutting head extends distally beyond the depth guide, positioning the cutting head adjacent the cutting route such that the cutting head extends distally beyond the cutting route for engagement with the patient's tissue, engaging the depth guide with a working surface of the recipient site cutting guide, wherein the working surface is spaced from the mounting surface, and wherein the cutting head extends distally beyond the mounting surface into contact with the patients' tissue when the depth guide is engaged with the working surface, causing the cutting head to rotate, and moving the cutting head in relation to the cutting route to form the recipient site.

In another aspect of the present disclosure, a method for grafting tissue is described that comprises forming a tissue graft having a non-circular configuration from donor tissue, and implanting the tissue graft into a recipient site having a corresponding non-circular configuration.

In certain embodiments, the present disclosure describes a tissue graft cutting guide/harvesting system that is suitable for creating non-circular tissue grafts from donor tissue. Additionally, systems, apparatus, and methods are described herein that may be suitable for creating a corresponding non-circular recipient site, e.g., socket, in a patient's tissue.

The recipient site cutting guide described herein may be selectively deformable by the user to allow the recipient site cutting guide to conform to the contour defined by the patient's anatomy (tissue). For example, the recipient site cutting guide may include sufficiently thin material, and/or by slots (reliefs), to increase flexibility, thus facilitating deformation.

During use, once the recipient site cutting guide is contoured to the patient's anatomy (tissue), it may be temporarily fixed thereto, for example, through the use of surgical pins, screws, nails, or the like.

The present disclosure further describes a cutting bit for use with the recipient site cutting guide that is capable of removing tissue to form the recipient site according to a specified, predetermined depth.

Following formation of the recipient site, the cutting guide is removed to expose the formed recipient site.

The present disclosure further describes a methodology, and related apparatus, for forming a tissue graft from donor tissue, e.g., an allograft, autograft, etc.

In certain embodiments, apparatus and methods are described pertaining to the formation of a tissue graft having a non-circular configuration corresponding to that of the recipient site, or a configuration that facilitates integration into the recipient site, e.g., by press fit, or other methods known in the art.

In certain embodiments, the tissue graft may be formed so as to define one or more protrusions, e.g., pegs, teeth, blocks, or other such projections, corresponding to one or more depressions, e.g., sockets, channels, or other such recesses, defined at the recipient site. For example, the tissue graft and the recipient site may each include multiple cuts at different angles that are adapted to engage one another in order to further facilitate securement of the tissue graft with respect to the recipient site.

In another aspect of the present disclosure, a cutting guide is disclosed for use during a surgical procedure to form a recipient site within a patient's tissue. The cutting guide comprises a deformable body that is reconfigurable to contour to the patient's tissue. The body includes proximal and distal surfaces, as well as a non-circular window through which the patient's tissue is accessed.

In certain embodiments, the body may include, e.g., be formed from, a flexible material. For example, the body may include a material selected from the group consisting of plastic, aluminum, stainless steel, titanium, and combinations thereof.

In certain embodiments, the body may include at least one weakened portion, e.g., at least one relief defining a plurality of teeth. In such embodiments, the teeth may define sidewalls extending transversely in relation to the proximal surface of the body, e.g., such that the sidewalls and the proximal surface of the body subtend an angle less than 90°, or such that the sidewalls and the proximal surface of the body extend in orthogonal relation.

In certain embodiments, the at least one weakened portion may include a first material, and remaining portions of the body may include a second material, wherein the first material is more flexible than the second material.

In certain embodiments, the body may further include a mounting portion configured and dimensioned to facilitate securement of the cutting guide in relation to the patient's tissue, e.g., at least one opening configured and dimensioned to receive a mounting member insertable into the patient's tissue through the at least one opening to secure the cutting guide in relation to the patient's tissue.

In certain embodiments, the body may further includes a shoulder extending inwardly into the window to define a cutting route, wherein the shoulder is engageable with a cutting bit such that the cutting bit follows the cutting route during formation of the recipient site.

In certain embodiments, the shoulder may be configured and dimensioned such that the cutting route defines a cutting area that is non-circular in configuration.

In certain embodiments, the shoulder may be configured and dimensioned such that the cutting area defines a length and a width, wherein the length is greater than the width.

In certain embodiments, the window may be enclosed, and the cutting route may be continuous in configuration. Alternatively, the window may be open such that the cutting route has defined, discrete endpoints.

In another aspect of the present disclosure, a system is disclosed for use during a surgical procedure to form a recipient site within a patient's tissue. The system comprises a cutting guide defining a cutting route, and a cutting bit configured and dimensioned for engagement with the cutting guide such that the cutting bit follows the cutting route during formation of the recipient site.

The cutting bit comprises a shaft having proximal and distal ends, and a cutting head positioned at a distal end of the shaft that is configured, dimensioned, and adapted to remove portions of the patient's tissue to thereby form the recipient site.

In certain embodiments, the cutting bit may further include a depth guide extending outwardly in relation to the shaft, and defining a distal surface configured and dimensioned for engagement with the cutting guide.

In certain embodiments, the depth guide may be configured as a sleeve positioned about the shaft.

In certain embodiments, the depth guide may be configured and dimensioned such that the cutting head extends distally beyond the cutting guide upon engagement of the distal surface of the depth guide with the cutting guide.

In certain embodiments, the cutting guide may include a body defining proximal and distal surfaces that is deformable such that the body is reconfigurable to contour to the patient's tissue.

In certain embodiments, the body may define a non-circular window through which the patient's tissue is accessed.

In certain embodiments, the body may further include a shoulder extending inwardly into the window, wherein the shoulder defines the cutting route, and is configured and dimensioned for engagement with the distal surface of the depth guide of the cutting bit.

In certain embodiments, the shoulder may be configured and dimensioned such that the cutting route defines a cutting area that is non-circular in configuration.

In certain embodiments, the shoulder may be configured and dimensioned such that the cutting area defines a length and a width, wherein the length is greater than the width.

In certain embodiments, the window may be enclosed. Alternatively, the window may be open such that the cutting route has defined, discrete endpoints.

In certain embodiments, the body may include, e.g., be formed from a flexible material. For example the body may include a material selected from the group consisting of plastic, aluminum, stainless steel, titanium, and combinations thereof.

In certain embodiments, the body may include at least one weakened portion, e.g., at least one relief defining a plurality of teeth. In such embodiments, the teeth may define sidewalls extending transversely in relation to the proximal surface of the body, e.g., such that the sidewalls and the proximal surface of the body subtend an angle less than 90°, or such that the sidewalls and the proximal surface of the body extend in orthogonal relation.

In certain embodiments, the at least one weakened portion may include a first material, and remaining portions of the body may include a second material, wherein the first material is more flexible than the second material.

In certain embodiments, the body may further include a mounting portion configured and dimensioned to facilitate securement of the cutting guide in relation to the patient's tissue, e.g., at least one opening configured and dimensioned to receive a mounting member insertable into the patient's tissue through the at least one opening to secure the cutting guide in relation to the patient's tissue.

In another aspect of the present disclosure, a cutting guide is disclosed for use in forming a tissue graft from donor tissue. The cutting guide comprises an upper body portion including a removable template defining a channel configured and dimensioned to receive a cutting implement such that the cutting implement is positionable within the channel in contact with the donor tissue, and a lower body portion separable from the upper body portion, wherein the upper and lower body portions collectively define an internal chamber configured and dimensioned to receive the donor tissue.

In certain embodiments, the template may include a pair of outwardly extending arms, and the upper body portion may include cleats configured and dimensioned to receive the arms such that the template is removable from the upper body portion for inversion and repositioning within the cleats.

In certain embodiments, the lower body portion may include retaining structure configured and dimensioned to inhibit relative movement between the donor tissue and the cutting guide, e.g., at least one opening configured and dimensioned to receive at least one fastener insertable into the donor tissue through the at least one opening.

In another aspect of the present disclosure, a method of forming a tissue graft from donor tissue is disclosed that comprises positioning the donor tissue within a cutting guide, inserting a cutting implement into a template of the cutting guide such that the cutting implement extends through the template into contact with the donor tissue, and moving the cutting implement through a channel defined by the template to remove portions of the donor tissue and thereby form the tissue graft.

In certain embodiments, positioning the donor tissue within the cutting guide may include positioning the donor tissue within an internal chamber collectively defined by upper and lower body portions of the cutting guide.

In certain embodiments, positioning the donor tissue within the internal chamber may include separating the upper and lower body portions of the cutting guide.

In certain embodiments, the method may further include removing the template from the cutting guide, inverting the template, and re-positioning the inverted template in the cutting guide.

In certain embodiments, removing the template from the cutting guide may include removing a pair of arms extending outwardly from the template from cleats defined by the cutting guide.

In certain embodiments, re-positioning the inverted template may include re-inserting the arms into the cleats.

In certain embodiments, the method may further include moving the cutting implement through the channel of the template following inversion of the template to remove additional portions of the donor tissue.

In certain embodiments, the method may further include securing the donor tissue in relation to the cutting guide.

In certain embodiments, securing the donor tissue in relation to the cutting guide may include inserting at least one fastener into the donor tissue through at least one corresponding opening formed in the cutting guide.

In another aspect of the present disclosure, a method of performing a surgical procedure is disclosed that comprises forming a tissue graft from donor tissue, forming a recipient site within a patient's tissue, and positioning the tissue graft within the recipient site.

Forming the tissue graft comprises: (i) positioning the donor tissue within a tissue graft cutting guide; (ii) inserting a cutting implement into a template of the tissue graft cutting guide such that the cutting implement extends through the template into contact with the donor tissue; and (iii) moving the cutting implement through a channel defined by the template to remove portions of the donor tissue and thereby form the tissue graft.

Forming the recipient site within the patient's tissue comprises: (i) deforming the recipient site cutting guide such that the recipient site cutting guide is contoured to the patient's tissue; (ii) positioning a cutting bit in engagement with the recipient site cutting guide; and (iii) moving the cutting bit in relation to the recipient site cutting guide such that the cutting bit follows a path defined by a cutting route of the recipient site cutting guide to thereby remove portions of the patient's tissue and form the recipient site.

In certain embodiments, deforming the recipient site cutting guide may include bending flexible material comprising a body of the recipient site cutting guide.

In certain embodiments, deforming the recipient site cutting guide may include bending a body of the recipient site cutting guide at a weakened portion of the body, e.g., a relief formed in the body.

In certain embodiments, positioning the method may further include positioning the recipient site cutting guide such that teeth defined by the relief contact the patient's tissue.

In certain embodiments, the method may further include securing the recipient site cutting guide in relation to the patient's tissue, e.g., by inserting a mounting member into the patient's tissue through at least one opening formed in the recipient site cutting guide.

In certain embodiments, positioning the cutting bit in engagement with the recipient site cutting guide may include positioning the cutting bit such that a cutting head of the cutting bit extends through a non-circular window of the recipient site cutting guide into contact with the patient's tissue.

In certain embodiments, positioning the cutting bit may include positioning the cutting bit in engagement with a shoulder extending inwardly into the window of the recipient site cutting guide, the shoulder defining the cutting route.

In certain embodiments, moving the cutting bit may include moving the cutting bit between discrete endpoints of the cutting route.

In certain embodiments, positioning the donor tissue within the tissue graft cutting guide may include positioning the donor tissue within an internal chamber collectively defined by upper and lower body portions of the tissue graft cutting guide.

In certain embodiments, positioning the donor tissue within the internal chamber may include separating the upper and lower body portions of the tissue graft cutting guide.

In certain embodiments, the method may further include removing the template from the upper body portion, inverting the template, and re-positioning the inverted template.

In certain embodiments, removing the template from the upper body portion may include removing a pair of arms extending outwardly from the template from cleats defined by the upper body portion.

In certain embodiments, re-positioning the template may include re-inserting the arms into the cleats.

In certain embodiments, the method may further include moving the cutting implement through the channel following inversion of the template to remove additional portions of the donor tissue.

In certain embodiments, the method may further include securing the donor tissue in relation to the tissue graft cutting guide.

In certain embodiments, securing the donor tissue in relation to the tissue graft cutting guide may include inserting at least one fastener into the donor tissue through at least one corresponding opening formed in the tissue graft cutting guide.

In another aspect of the present disclosure, a method is described for forming a recipient site within a patient's tissue that is configured and dimensioned to receive a tissue graft. The method comprises deforming a recipient site cutting guide such that the recipient site cutting guide is contoured to the patient's tissue, positioning a cutting bit in engagement with the recipient site cutting guide, and moving the cutting bit in relation to the recipient site cutting guide such that the cutting bit follows a cutting route defined by the recipient site cutting guide to thereby remove portions of the patient's tissue and form the recipient site.

In certain embodiments, deforming the recipient site cutting guide may include bending flexible material comprising a body of the recipient site cutting guide.

In certain embodiments, deforming the recipient site cutting guide may include bending a body of the recipient site cutting guide at a weakened portion of the body, e.g., at a relief formed in the body.

In certain embodiments, the method may further include positioning the recipient site cutting guide in relation to the patient's tissue such that teeth defined by the relief contact the patient's tissue.

In certain embodiments, the method may further include securing the recipient site cutting guide in relation to the patient's tissue, e.g., by inserting a mounting member into the patient's tissue through at least one opening formed in the recipient site cutting guide.

In certain embodiments, positioning the cutting bit in engagement with the recipient site cutting guide may include positioning the cutting bit such that a cutting head of the cutting bit extends through a non-circular window of the recipient site cutting guide to facilitate contact with the patient's tissue.

In certain embodiments, positioning the cutting bit may include positioning the cutting bit in engagement with a shoulder extending inwardly into the window of the recipient site cutting guide that defines the cutting route.

In certain embodiments, positioning the cutting bit in engagement with the shoulder may include positioning a depth guide of the cutting bit in engagement with the shoulder.

In certain embodiments, moving the cutting bit in relation to the recipient site cutting guide may include moving the depth guide along the shoulder.

In certain embodiments, moving the cutting bit in relation to the recipient site cutting guide may include moving the cutting bit between discrete endpoints of the cutting route.

Other objects, features, and advantages of the present disclosure will become apparent with reference to the drawings and detailed description of the illustrative embodiments that follow.

### BRIEF DESCRIPTION OF THE DRAWINGS

FIG. 1 is a top, perspective view of a recipient site in a patient's tissue formed according to the principles of the present disclosure;

FIG. 2 is a top, perspective view of a tissue graft for positioning with the recipient site seen in FIG. 1 formed according to the principles of the present disclosure;

FIG. 3 is a side, elevational view of a system including a recipient site cutting guide and a cutting bit (shown assembled) for use in forming the recipient site seen in FIG. 1;

FIG. 4 is a side, elevational view of the recipient site cutting guide and the cutting bit seen in FIG. 3 (shown separated);

FIG. 5 is a top, plan view of the recipient site cutting guide together with the patient's tissue;

FIG. 6 is a side, elevational view of the recipient site cutting guide together with the patient's tissue;

FIG. 7A is a top, plan view of the recipient site cutting guide;

FIG. 7B is a top, plan view of the recipient site cutting guide together with the patient's tissue;

FIG. 8 is a side, elevational view of an alternate embodiment of the recipient site cutting guide together with the patient's tissue;

FIG. 9 is a top, plan view of an alternate embodiment of the recipient site cutting guide;

FIG. 10 is a top, plan view of the embodiment of the recipient site cutting guide seen in FIG. 9 together with the patient's tissue;

FIG. 11 is a top, perspective view of a tissue graft cutting guide including a shaping template for use in forming the tissue graft seen in FIG. 2;

FIG. 12 is a top, perspective view of the tissue graft cutting guide seen in FIG. 11 including an alternate embodiment of the shaping template prior to inversion; and

FIG. 13 is a top, perspective view of the tissue graft cutting guide seen in FIG. 12 subsequent to inversion of the shaping template.

### DETAILED DESCRIPTION

In the following detailed description, reference is made to the accompanying drawings, which depict non-limiting, illustrative embodiments of the present disclosure. Other embodiments may be utilized and logical structural, mechanical, electrical, and chemical changes may be made without departing from the scope of the present disclosure. To avoid unnecessary detail, the following description may omit certain information, items, or details known to those skilled in the art. The following detailed description is provided without limitation, the scope of which is defined by the appended claims.

Throughout the present disclosure, the term “tissue” should be understood as including various forms of biological structural material, including, but not limited to, bone and cartilage. Additionally, the terms “height,” “width,” “thickness,” “above,” “below,” “closer,” “further,” and any variation(s) thereof, are used herein in a relative capacity, and are intended to be interpreted in accordance with the perspective shown in the corresponding figure(s). Additionally, as used herein, the term “distal,” and variations thereof, should be understood as referring to that portion of a structure, or to a direction, that is furthest from the user, whereas the term “proximal,” and variations thereof, should be understood as referring to that portion of a structure, or to a direction, that is closest from the user. Moreover, the term “cutting,” and variations thereof, should be understood as referring to the process by which tissue is removed, and not necessarily as requiring an incisive or sharpened surface. Furthermore, the term “bit,” e.g., “cutting bit,” should be understood to encompass any implement suitable for the intended purpose of removing tissue.

This specification relates to systems, apparatus, and methods that may be adapted, in certain embodiments, to create a non-circular tissue graft, and a corresponding non-circular recipient site in tissue, e.g., a socket, for receiving the tissue graft. The non-circular tissue grafts described herein can be used, for example, in OCA surgery to repair relatively large defects, and may encompass allografts, autografts, and the like. The non-circular tissue grafts, recipient sites, and related methodologies described herein provide a number of benefits over those which are known in the art. For example, the non-circular tissue grafts, recipient sites, and related methodologies described herein increase the preservation of native tissue when compared with known techniques, and are capable of demonstrating significantly better fixation stability when compared to known circular counterparts, wherein two or more circular tissue grafts are typically required per joint surface. Moreover, the non-circular tissue grafts, recipient sites, and related methodologies described herein allow a significantly larger amount of healthy tissue to be maintained in patients with high aspect ratio defects (e.g., defects having a length of more than 1.5 times their width) when compared to the use of circular tissue grafts, recipient sites, and related methodologies.

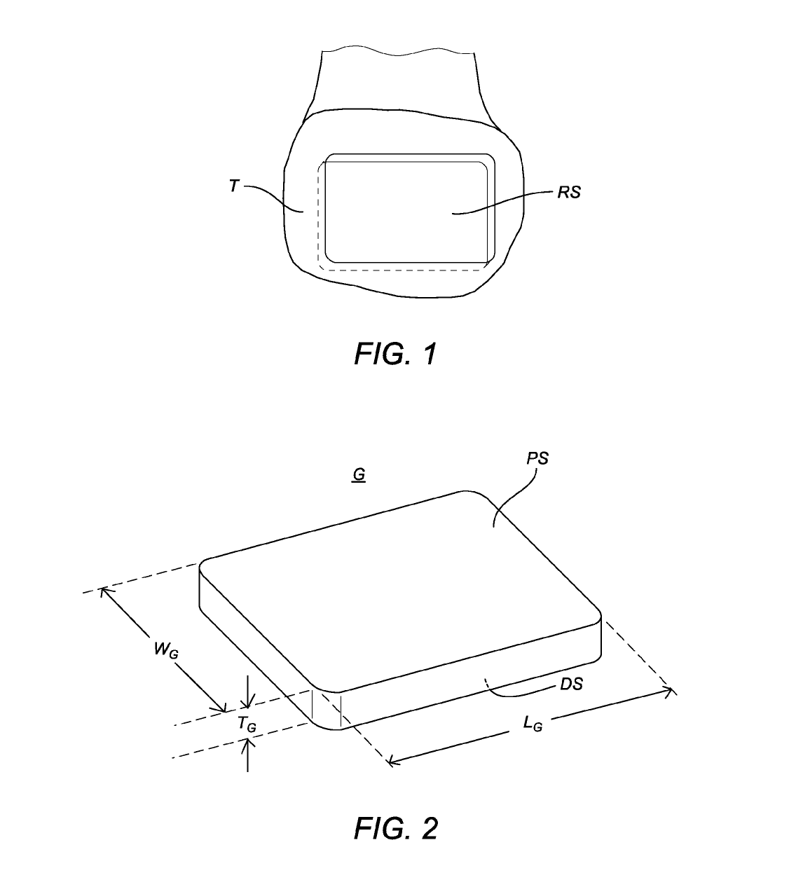

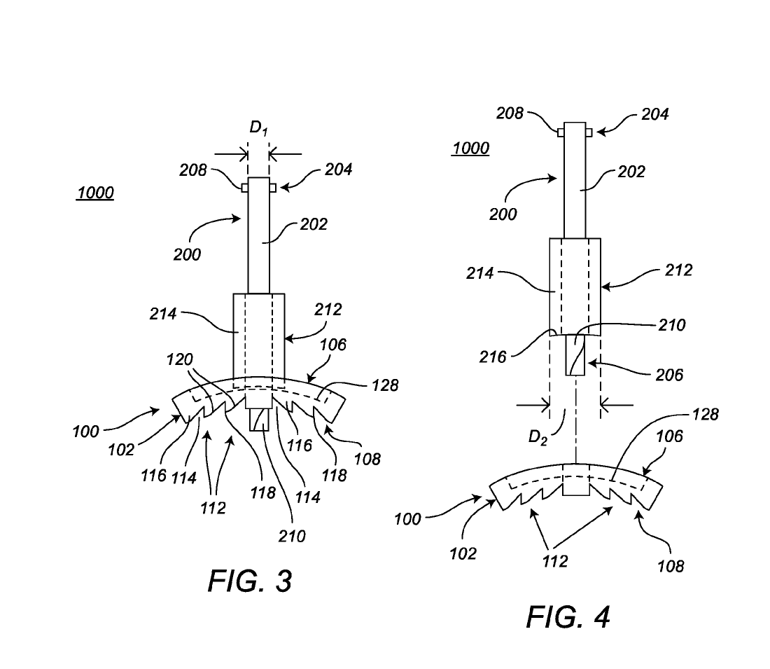

With reference to FIGS. 1-7B, a system 1000 is disclosed useful in the formation of a recipient site RS (FIG. 1), e.g., a cavity, socket, or the like, in tissue T that is configured and dimensioned to receive a corresponding tissue graft G (FIG. 2). The system 1000 includes a recipient site cutting guide 100 FIGS. 3, 4), and a cutting bit 200 for use with the cutting guide 100. Also described are methods for forming the recipient site RS (FIG. 1) using the cutting guide 100 and the cutting bit 200.

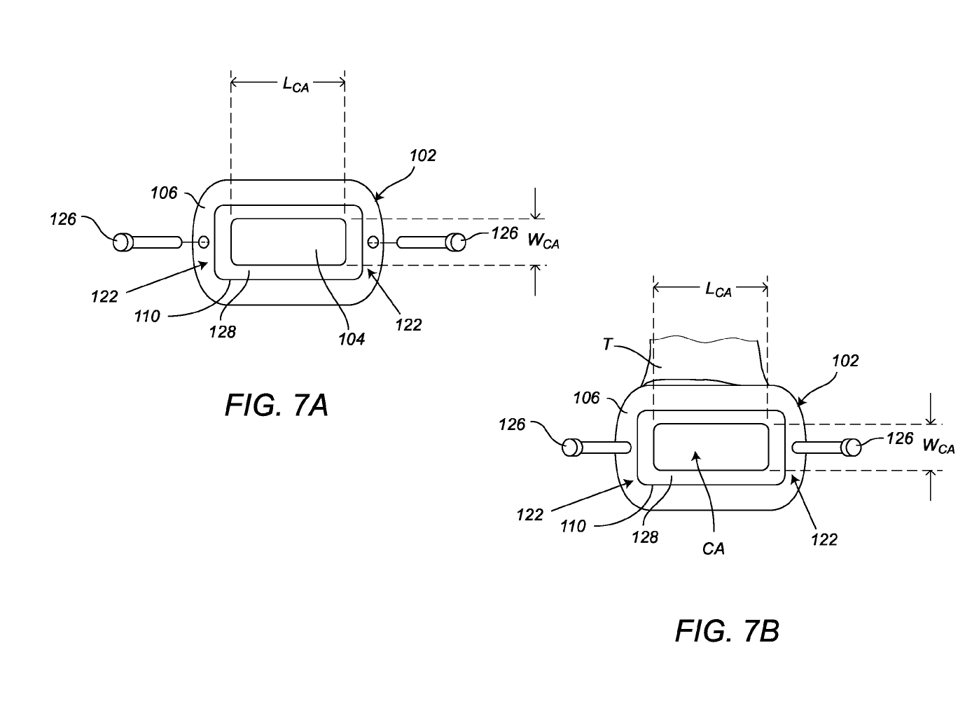

The cutting guide 100 includes a body 102 (FIGS. 3-7B) which may define any desired geometrical configuration, dependent upon the requirements of a particular surgical procedure. For example, it is envisioned that the body 102 of the cutting guide 100 may be non-circular, e.g., rectangular, as illustrated in FIGS. 3-6, ovoid, elliptical, triangular, etc. The body 102 defines a window 104 (FIG. 7A), through which, access to the tissue T is obtained, as well as a proximal (working) surface 106, a distal (mounting) surface 108, and a guide wall 110 that extends between the surfaces 106, 108 such that the surface 106 is separated from the surface 108 by a distance equal to the height of the guide wall 110.

It is envisioned that the surfaces 106, 108 may extend in non-intersecting planes, as illustrated in the embodiment depicted in FIGS. 3-7B, for example, or alternatively, that the planes along which the surfaces 102, 104 extend may intersect.

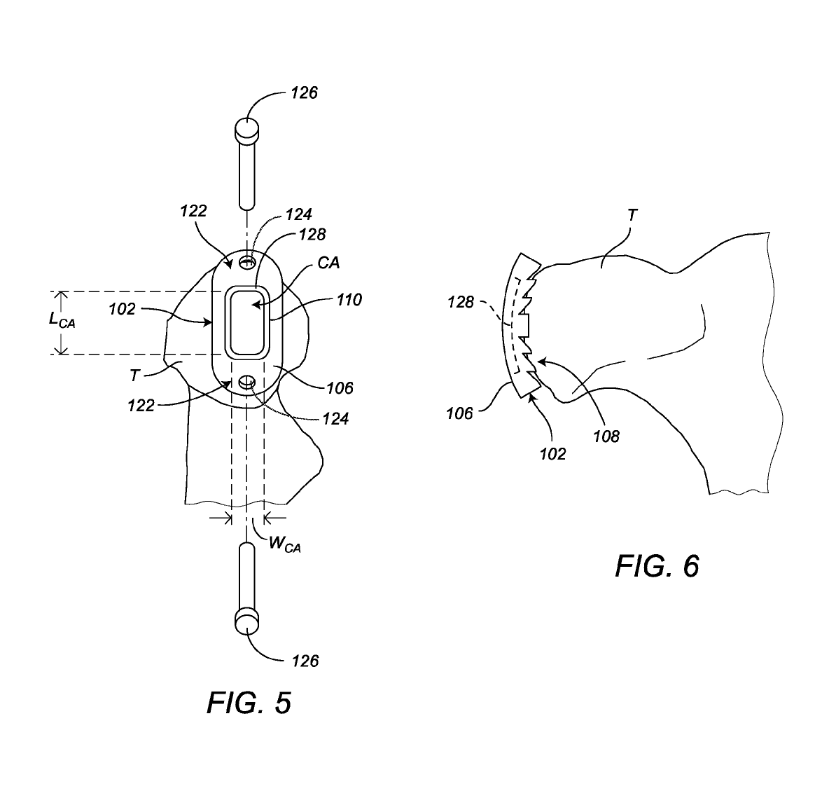

It is further envisioned that the guide wall 110 may include curvate portions, as seen in FIGS. 5, 7A, and 7B, for example, or that the guide wall 110 may include only linear portions.

In certain embodiments, the cutting guide 100 may be selectively deformable to permit the cutting guide 100 to contour to the tissue T. For example, the cutting guide 100 may include, e.g., be formed from, a deformable material compatible with surgical practice, such as, for example, plastic, polymers, aluminum, stainless steel, titanium, or combinations thereof. Additionally, or alternatively, the cutting guide 100 may include one or more weakened portions 112 (FIGS. 3, 4) to facilitate the controlled deformation thereof. For example, as illustrated in FIGS. 3 and 4, the cutting guide 100 may include one or more reliefs 114, i.e., areas of reduced thickness, defining one or more teeth 116 forming contact points 118, i.e., locations where the cutting guide 100 contacts the tissue T, to increase flexibility in the cutting guide 100, and the ability of the cutting guide 100 to be contoured to the tissue T.

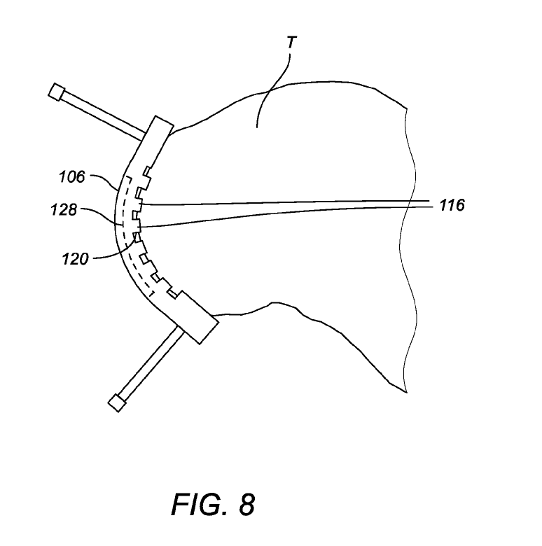

The teeth 116 include sidewalls 120 that extend in transverse relation to the proximal surface 106. In certain embodiments, such as that illustrated in FIGS. 3 and 4, it is envisioned that the sidewalls 120 may extend along axes forming an angle of less than 90° with the proximal surface 106 to reduce the surface area of the contact points 118, and thereby increase friction between the cutting guide 100 and the tissue T. Alternatively, it is envisioned that the sidewalls 120 may extend along axes that are orthogonal in relation to the proximal surface 106, as illustrated in FIG. 8.

Additionally, or alternatively, it is envisioned that the weakened portions 112 may include, e.g., be formed from, a material of lower rigidity than the remainder of the cutting guide 100 to further increase flexibility and deformability of the cutting guide 100.

In certain embodiments, the cutting guide 100 may also include one or more mounting portions 122 (FIGS. 5, 7A, 7B) configured and dimensioned to facilitate connection of the cutting guide 100 to the tissue T. For example, the mounting portion(s) 122 may include one or more eyelets 124, or other such openings, extending through the mounting portion(s) 122 that are configured and dimensioned to receive one or more mounting members 126. Although illustrated as pins in FIGS. 5, 7A, and 7B, the mounting member(s) 126 may be any structure suitable for the intended purpose of securing the cutting guide 100 in relation to the tissue T, e.g., screws, nails, or the like.

After positioning the cutting guide 100 adjacent the tissue T as desired, e.g., after deforming and contouring the cutting guide 100 to the tissue T, the cutting guide 100 can be fixed in relation to the tissue T by passing the mounting member(s) 126 through the eyelet(s) 124 into the tissue T.

With continued reference to FIGS. 5, 7A, and 7B, the body 102 of the cutting guide 100 may further include a shoulder 128 that extends inwardly into the window 104, e.g., in a plane that extends in substantially parallel relation to the proximal surface 106, for example, at an angle between 0° and 20° to define a cutting route. As illustrated in FIGS. 5, 7A, and 7B, it is envisioned that the shoulder 128 may extend inwardly from the guide wall 110, or alternatively, from the proximal surface 106 or the distal surface 108.

The shoulder 128 defines a cutting route which may have a configuration identical to that defined by the body 102 of the cutting guide 100, e.g., a non-circular configuration, or alternatively, a configuration that deviates therefrom. The shoulder 128 defines a cutting area CA (FIGS. 5, 7B) within the window 104 (FIG. 7A) having a length L_{CA} and a width W_{CA}, wherein the length L_{CA} may be greater than the width W_{CA}, the width W_{CA} may be greater than the length L_{CA}, or wherein the length L_{CA} and the width W_{CA} may be equal to each other. For example, the cutting area CA may have an aspect ratio (L_{CA}:W_{CA}), i.e., the ratio of the length L_{CA} of the cutting area CA to the width W_{CA} of the cutting area CA, greater than 1.0, e.g., 1.5.

With reference now to FIGS. 3 and 4 in particular, the cutting bit 200 will be described. The cutting bit 200 includes an elongate shaft 202 having a proximal (first) end 204 and a distal (second) end 206 defining a length therebetween, and defines an external diameter D₁. The cutting bit 200 may further comprise an arbor 208, or other such surface irregularity, e.g., a projection or a recess, to facilitate connection of the cutting bit 200 to a source of power (not shown), such as a motor, drill, or other such tool.

The cutting bit 200 further includes a cutting head 210 spaced from the first end 204 of the shaft 202 that is configured and dimensioned to penetrate and remove material from the tissue T (FIG. 1).

In certain embodiments, such as that illustrated in FIGS. 3 and 4, for example, the cutting bit 200 may further include a depth guide 212 positioned on the shaft 202 that defines an external diameter D₂, which may be larger than the external diameter D₁ of the elongate shaft 202, as illustrated in FIGS. 3 and 4. In certain embodiments, the depth guide 212 may be configured as a sleeve 214, as illustrated in FIGS. 3 and 4, or a collar (not shown). The depth guide 212 defines a distal surface 216 (FIG. 4) that is configured and dimensioned to contact the shoulder 128 (FIGS. 3-5, 7A, 7B) of the cutting guide 100. The distal surface 216 may be either planar in configuration, or alternatively, may include one or more arcuate portions dependent upon the particular configuration of the shoulder 128.

It is envisioned that the depth guide 212 may be releasably secured to the elongate shaft 202 such that the depth guide 212 is repositionable along the elongate shaft 202. For example, the depth guide 212 may include a set screw (not shown) extending therethrough that can be moved into contact with the elongate shaft 202 to thereby fix the position of the depth guide 212 in relation to the elongate shaft 202.

The cutting bit 200 is dimensioned such that the cutting head 210 extends beyond the cutting guide 100 when assembled together, as illustrated in FIG. 3, to facilitate the removal of material from the tissue T, and thus, formation of the recipient site RS (FIG. 2).

With reference now to FIGS. 1-7B, use of the system 1000 will be described during formation of the recipient site RS (FIG. 1). Initially, if necessary, or desirable, the configuration of the cutting guide 100 can be manipulated to conform to the contour defined by the patient's anatomy, as seen in FIG. 6. Specifically, the cutting guide 100 is positioned such that the distal surface 108 is oriented towards the tissue T, and the proximal surface 106 is oriented away from the tissue T. The cutting guide 100 may then be fixed relative to the tissue T, e.g., via insertion of the mounting member(s) 126 (FIGS. 5, 7A, 7B) into the tissue T via the mounting eyelet(s) 124.

The cutting bit 200 can then be advanced distally into contact with the tissue T until the engaging surface 214 (FIG. 4) of the depth guide 212 (FIGS. 3, 4) contacts the shoulder 128, as illustrated in FIG. 3, whereby the distance between the cutting head 210 and the cutting guide 100 can be held constant to thereby facilitate formation the of a recipient site RS having a consistent depth. In those embodiments wherein the depth guide 212 is movable in relation to the elongate shaft 202 of the cutting bit 200, the position of the depth guide 212 in relation to the cutting head 210 can be varied to create the recipient site RS with any desirable depth, either constant or variable (via proximal and distal movement of the cutting bit 200).

During formation of the recipient site RS (FIG. 1), the cutting bit 200 is moved in relation to the cutting guide 100, e.g., such that the distal surface 216 of the depth guide 212 slides along the shoulder 128, tracing the configuration of the shoulder 128 and following the cutting route, whereby the recipient site RS is formed in correspondence with the configuration and dimensions of the shoulder 128.

In certain methods of use, the recipient site RS (FIG. 1) may be formed so as to define one or more depressions (not shown), e.g., cavities, channels, or other such recesses, via proximal and distal movement of the cutting bit 200 in relation the cutting guide 100. Additionally, or alternatively, the recipient site RS may be formed so as to define one or more angled cuts, either via use of the cutting bit 200, or a separate cutting implement, e.g., a scalpel.

Following formation of the recipient site RS, the cutting bit 200 can be separated from the cutting guide 100, the cutting guide 100 can be removed from the tissue T, and the tissue graft G (FIG. 2) can be placed at the recipient site RS.

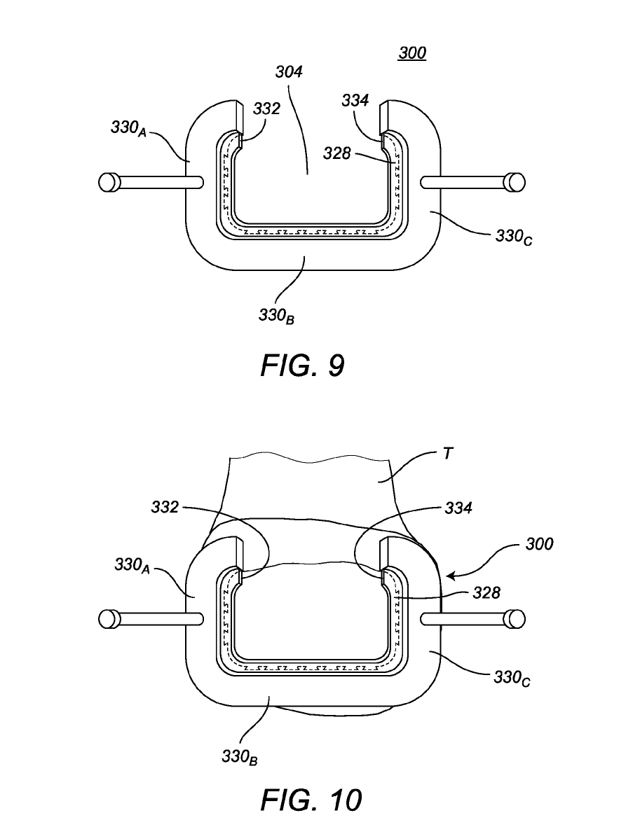

With reference now to FIGS. 9 and 10, an alternate embodiment of the cutting guide, identified by the reference character 300, will be discussed. The cutting guide 300 is identical to the cutting guide 100 discussed above in connection with FIGS. 3-7B but for any distinctions that are specifically noted. Accordingly, a discussion of certain features common to the cutting guides 100, 300 may be omitted in the interest of brevity.

Whereas the cutting guide 100 defines an enclosed window 104 (FIG. 7A), the window 304 defined by the cutting guide 300 is open. For example, in the embodiment of the cutting guide 300 shown in FIGS. 9 and 10, the window 304 is defined by three segments 330_{A}, 330_{B}, 330_{C}, and thus includes a shoulder 328 defining a discontinuous cutting route with defined, discrete endpoints 332, 334. In the specifically embodiment illustrated in FIGS. 9 and 10, for example, the shoulder 328, and thus, the cutting route, is generally U-shaped in configuration.

The open configuration of the window 304 permits lateral installation of the tissue graft G (FIG. 2), i.e., parallel to a joint surface, as opposed to vertical installation i.e., perpendicular to the joint surface.

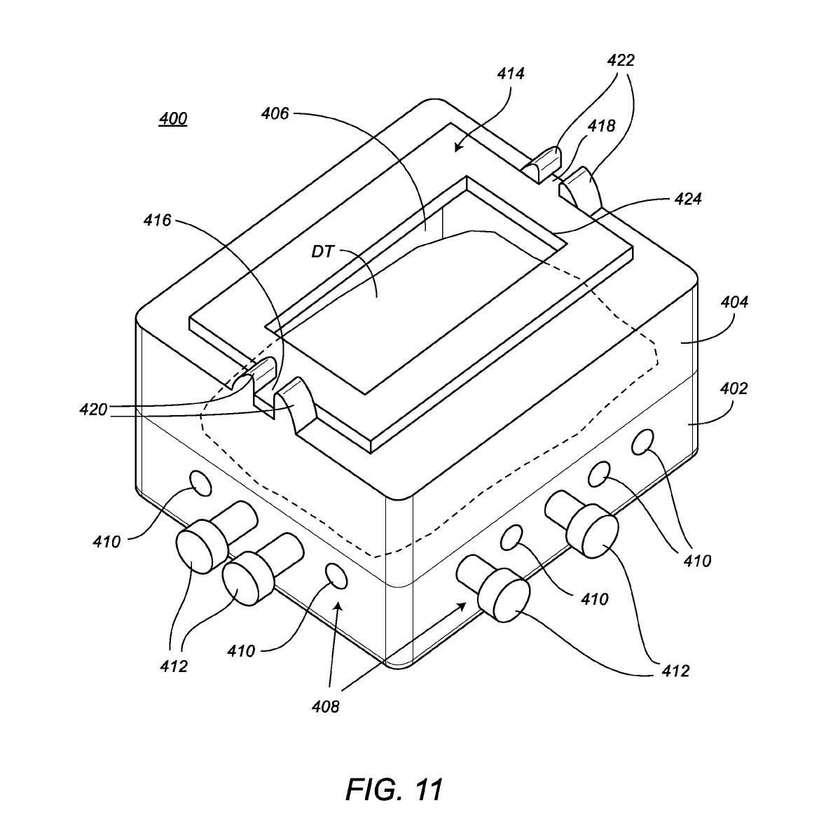

Referring now to FIG. 11, a cutting guide 400 will be discussed for use in forming the tissue graft G (FIG. 2) from a block of donor tissue DT, e.g., allograft tissue, autograft tissue, etc.

Dependent upon the requirements of the particular surgical procedure in which the cutting guide 400 is employed, the tissue graft G may be formed so as to realize a rectangular configuration, an ovoid configuration, an elliptical configuration, a triangular configuration, etc., or combinations thereof.

Referring briefly to FIG. 2, an exemplary tissue graft G is illustrated that may be formed using the tissue graft cutting guide 400. The tissue graft G is non-circular in configuration, and defines a length L_{G}, a width W_{G}, and a thickness T_{G}. As illustrated in FIG. 2, it is envisioned that the tissue graft G may be formed such that the length L_{G} is greater than the width W_{G}.

The tissue graft G further comprises a proximal (exposed) surface PS, and a distal (application) surface DS, wherein the proximal surface PS is separated from the distal surface DS by a distance equal to the thickness T_{G} of the tissue graft G. In certain embodiments, it is envisioned that the cutting guide 400 may be configured and dimensioned to form the tissue graft G such that the respective proximal and distal surfaces PS, DS of the tissue graft G extend in non-intersecting planes, or alternatively, that the planes along which the surfaces PS, DS extend may intersect.

Referring again to FIG. 11, the tissue graft cutting guide 400 includes separable lower and upper body portions 402, 404, respectively, that collectively define an internal chamber 406 configured and dimensioned to receive the donor tissue DT. In one embodiment, such as that illustrated in FIG. 11, the cutting guide 400 may include retaining structure 408 to inhibit movement of the donor tissue DT within the internal chamber 406 during cutting. Specifically, in embodiment illustrated in FIG. 11, the lower body portion 402 includes one or more openings 410 that are configured and dimensioned to receive fasteners 412, which extend through the lower body portion 402 into contact with the donor tissue DT to secure the donor tissue DT in relation to the cutting guide 400. Although depicted as pins in the illustrated embodiment, the fasteners 412 may be any structure suitable for the intended purpose of inhibiting movement of the donor tissue DT within the internal chamber 406 during cutting, e.g., screws, nails, or the like.

The upper body portion 404 of the tissue graft cutting guide 400 includes a removable shaping template 414. For example in the embodiment illustrated in FIG. 11, the shaping template 414 includes a pair of outwardly extending arms 416, 418, and the upper body portion 404 includes a pair of cleats 420, 422 that removably receive the arms 416, 418, respectively, e.g., in snap-fit relation. The shaping template 414 defines a channel (opening) 424 that is configured and dimensioned to receive a cutting implement (not shown), e.g., a scalpel, routing tool, or the like. To form the tissue graft G in correspondence with the recipient site RS (FIG. 1), the channel 424 is configured and dimensioned in correspondence with the shoulder 128 (FIGS. 5, 7A, 7B) of the recipient site cutting guide 100 defining the cutting route. In the embodiment illustrated in FIG. 11, for example, the channel 424 and the cutting route defined by the shoulder 128 are each non-circular, and generally rectangular in configuration.

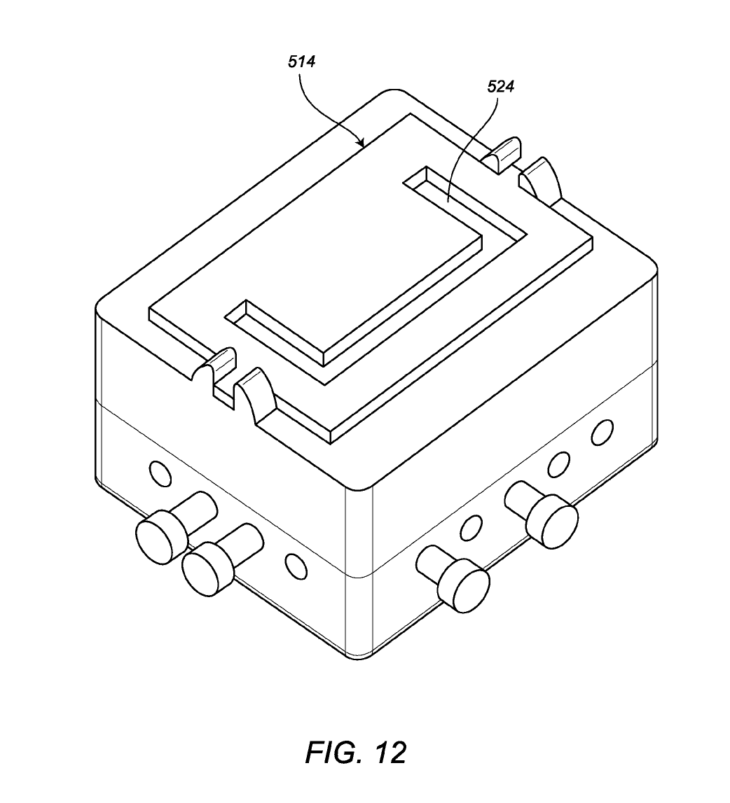

Although illustrated as generally rectangular in the embodiment illustrated in FIG. 11, the channel 424 of the shaping template 414, and the cutting route defined by the shoulder 128, may define alternate configurations in additional embodiments of the present disclosure. For example, with reference FIG. 12, an alternate embodiment of the template is illustrated, identified by the reference character 514, for use with the tissue graft cutting guide 300 (FIGS. 9, 10). As seen in FIG. 12, the shaping template 514 includes a channel 524 defining a (generally U-shaped) configuration and dimensions corresponding to the configuration and dimensions of the shoulder 328 defining the cutting route.

Other configurations are also envisioned. For example, the channel 424 (and the cutting route defined by the shoulder 128) may be generally c-shaped, oval, triangular, polygonal, etc., and may include linear and non-linear portions. It is also envisioned that the tissue graft cutting guide 400 may be provided with a series of templates 414, each including a channel 424 with a different configuration, to further increase adaptability of the tissue graft cutting guide 400, and the ability to create the tissue graft G (FIG. 2) according to any desired configuration.

With reference again to FIG. 11, a method of forming the tissue graft G (FIG. 2) using the tissue graft cutting guide 400 will be discussed. Initially, the body portions 402, 404 are separated, the donor tissue DT is positioned within the internal chamber 406, and the internal chamber 406 is closed by returning the body portions 402, 404 to the position illustrated in FIG. 11. Thereafter, if necessary or desirable, the retaining structure 408 may be utilized to secure the donor tissue DT within the internal chamber 406. For example, in the embodiment illustrated in FIG. 11, the fastener(s) 412 may be inserted into the donor tissue DT through the opening(s) 410.

If multiple templates 414 are provided, an appropriate shaping template 414 is selected, and is secured to the upper body portion 404, e.g., by positioning the arms 418, 418 within the cleats 420, 422. A cutting implement is then placed into contact with the donor tissue DT, through the channel 424, and is moved along the path defined by the channel 424 to remove material from the donor tissue DT, and thereby form the tissue graft G (FIG. 2).

In an alternate method of use, multiple shaping templates 414 may be employed. For example, a first shaping template 414 may be used to initially remove certain portions of the donor tissue DT which can be thereafter substituted for one or more different templates, e.g., templates including channels 424 having different configurations and/or dimensions, to remove additional portions of the donor tissue DT.

After formation of the tissue graft G, the tissue graft G is placed at the recipient site RS (FIG. 1). Thereafter, if necessary or desirable, the tissue graft G can be attached to the recipient site RS using either temporary or permanent attachment structures (not shown), e.g., fixation screws, bone plates, or the like.

In certain embodiments, and methods of use, the tissue graft G may be formed so as to define one or more surface irregularities, e.g., protrusions, pegs, teeth, blocks, angled cuts, etc., for mating engagement with corresponding surface irregularities at the recipient site RS (FIG. 1). The inclusion of such surface irregularities in the tissue graft G and the recipient site RS may serve to increase friction between the tissue graft G and the recipient site RS, and thus, stability of the tissue graft G following placement at the recipient site RS.

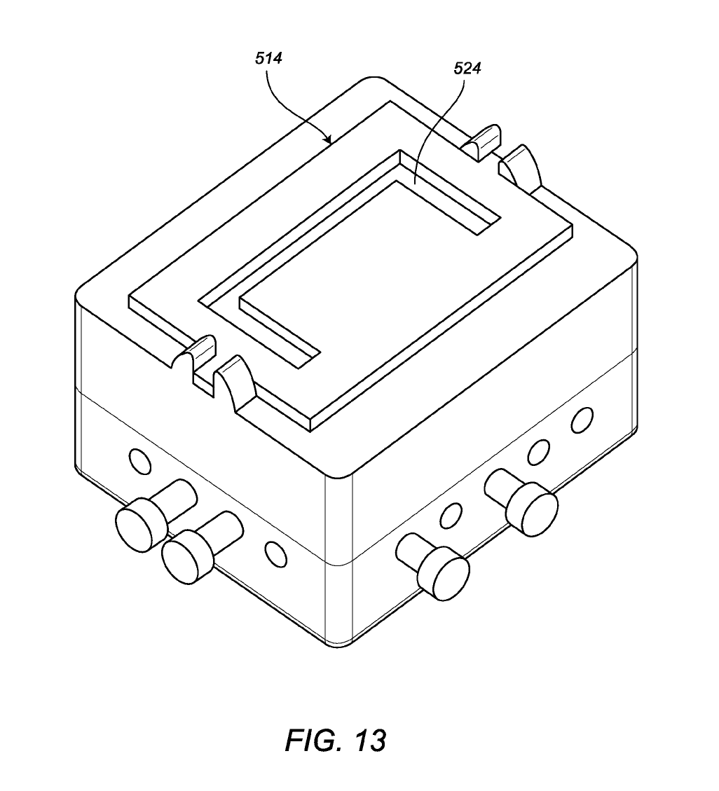

With reference to FIGS. 12 and 13, in another method of use, e.g., where the tissue graft G (FIG. 2) and the recipient site RS (FIG. 1) are generally symmetrical in configuration, it is envisioned that the shaping template 514 may be utilized to initially remove certain portions of the donor tissue DT, and thereafter, that the shaping template 514 can be removed, inverted, and replaced to facilitate the removal of additional portions of the donor tissue DT. For example, in the instance where the tissue graft G and the recipient site RS are to be formed so as to realize generally rectangular configurations, as illustrated in FIGS. 1 and 2, an initial cut (or cuts) can be made through the channel 524 with the shaping template 514 positioned as illustrated in FIG. 12. Thereafter, the shaping template 514 can be removed, inverted, and replaced, as illustrated in FIG. 13, such that a subsequent cut (or cuts) can be made to remove additional portions of the donor tissue DT (FIG. 11), and thereby form the tissue graft G (FIG. 2) in correspondence with the recipient site RS (FIG. 1).

While the present disclosure has been described in connection with specific, illustrative embodiments, it should be understood that the subject matter of the present disclosure is capable of further modifications. For example, persons skilled in the art will understand that additional components and features may be added to any of the embodiments discussed herein above, and that those elements and features described in connection with any one embodiment may also be applicable to, or combined with, those of any other embodiment without departing from the scope of the present disclosure.

The scope of the present disclosure is intended to cover any variations, uses, and/or adaptations of the presently disclosed subject matter in accordance with the principles of the present disclosure, including such departures from the present disclosure as come within known or customary practice within the art to which the present disclosure pertains, and as may be applied to the elements, components, and features set forth herein above.

1. A cutting guide for use during a surgical procedure to form a recipient site within a patient's tissue, the cutting guide comprising a body defining proximal and distal surfaces, the body being deformable such that the body is reconfigurable to contour to the patient's tissue, the body defining a non-circular window through which the patient's tissue is accessed.

2. The cutting guide of claim 1, wherein the body includes a flexible material.

3. The cutting guide of claim 2, wherein the body includes a material selected from the group consisting of plastic, aluminum, stainless steel, titanium, and combinations thereof.

4. The cutting guide of claim 1, wherein the body includes at least one weakened portion.

5. The cutting guide of claim 4, wherein the at least one weakened portion includes at least one relief formed in the body, the at least one relief defining a plurality of teeth.

6. The cutting guide of claim 5, wherein the teeth define sidewalls extending transversely in relation to the proximal surface of the body.

7. The cutting guide of claim 6, wherein the sidewalls and the proximal surface of the body subtend an angle less than 90°.

8. The cutting guide of claim 6, wherein the sidewalls and the proximal surface of the body extend in orthogonal relation.

9. The cutting guide of claim 4, wherein the at least one weakened portion of the body includes a first material, and remaining portions of the body include a second material, the first material being more flexible than the second material.

10. The cutting guide of claim 1, wherein the body further includes a mounting portion configured and dimensioned to facilitate securement of the cutting guide in relation to the patient's tissue.

11. The cutting guide of claim 10, wherein the mounting portion includes at least one opening configured and dimensioned to receive a mounting member insertable into the patient's tissue through the at least one opening to secure the cutting guide in relation to the patient's tissue.

12. The cutting guide of claim 1, wherein the body further includes a shoulder extending inwardly into the window to define a cutting route, the shoulder being engageable with a cutting bit such that the cutting bit follows the cutting route during formation of the recipient site.

13. The cutting guide of claim 12, wherein the shoulder is configured and dimensioned such that the cutting route defines a cutting area that is non-circular in configuration.

14. The cutting guide of claim 13, wherein the shoulder is configured and dimensioned such that the cutting area defines a length and a width, the length being greater than the width.

15. The cutting guide of claim 12, wherein the window is enclosed, and the cutting route is continuous in configuration.

16. The cutting guide of claim 12, wherein the window is open, and the cutting route has defined, discrete endpoints.

17. A system for use during a surgical procedure to form a recipient site within a patient's tissue, the system comprising:
a cutting guide defining a cutting route; and
a cutting bit configured and dimensioned for engagement with the cutting guide such that the cutting bit follows the cutting route during formation of the recipient site, the cutting bit comprising:
a shaft having proximal and distal ends; and
a cutting head positioned at a distal end of the shaft, the cutting head being configured, dimensioned, and adapted to remove portions of the patient's tissue to thereby form the recipient site.

18. The system of claim 17, wherein the cutting bit further includes a depth guide extending outwardly in relation to the shaft, the depth guide defining a distal surface configured and dimensioned for engagement with the cutting guide.

19. The system of claim 18, wherein the depth guide is configured as a sleeve positioned about the shaft.

20. The system of claim 18, wherein the depth guide is configured and dimensioned such that the cutting head extends distally beyond the cutting guide upon engagement of the distal surface of the depth guide with the cutting guide.

21. The system of claim 20, wherein the cutting guide includes a body defining proximal and distal surfaces, the body being deformable such that the body is reconfigurable to contour to the patient's tissue.

22. The system of claim 21, wherein the body defines a non-circular window through which the patient's tissue is accessed.

23. The system of claim 22, wherein the body further includes a shoulder extending inwardly into the window, the shoulder defining the cutting route, and being configured and dimensioned for engagement with the distal surface of the depth guide of the cutting bit.

24. The system of claim 23, wherein the shoulder is configured and dimensioned such that the cutting route defines a cutting area that is non-circular in configuration.

25. The system of claim 24, wherein the shoulder is configured and dimensioned such that the cutting area defines a length and a width, the length being greater than the width.

26. The cutting guide of claim 22, wherein the window is enclosed.

27. The cutting guide of claim 22, wherein the window is open, and the cutting route has defined, discrete endpoints.

28. The system of claim 21, wherein the body includes a flexible material.

29. The system of claim 28, wherein the body includes a material selected from the group consisting of plastic, aluminum, stainless steel, titanium, and combinations thereof.

30. The system of claim 21, wherein the body includes at least one weakened portion.

31. The system of claim 30, wherein the at least one weakened portion includes at least one relief formed in the body, the at least one relief defining a plurality of teeth.

32. The system of claim 31, wherein the teeth define sidewalls extending transversely in relation to the proximal surface of the body.

33. The system of claim 32, wherein the sidewalls and the proximal surface of the body subtend an angle less than 90°.

34. The system of claim 32, wherein the sidewalls and the proximal surface of the body extend in orthogonal relation.

35. The system of claim 30, wherein the at least one weakened portion includes a first material, and remaining portions of the body include a second material, the first material being more flexible than the second material.

36. The system of claim 21, wherein the body further includes a mounting portion configured and dimensioned to facilitate securement of the cutting guide in relation to the patient's tissue.

37. The system of claim 36, wherein the mounting portion includes at least one opening configured and dimensioned to receive a mounting member insertable into the patient's tissue through the at least one opening to secure the cutting guide in relation to the patient's tissue.

38. A cutting guide for use in forming a tissue graft from donor tissue, the cutting guide comprising:
an upper body portion including a removable template, the template defining a channel configured and dimensioned to receive a cutting implement such that the cutting implement is positionable within the channel in contact with the donor tissue; and
a lower body portion separable from the upper body portion, the upper and lower body portions collectively defining an internal chamber configured and dimensioned to receive the donor tissue.

39. The cutting guide of claim 38, wherein the template includes a pair of outwardly extending arms, and the upper body portion includes cleats configured and dimensioned to receive the arms such that the template is removable from the upper body portion for inversion and repositioning within the cleats.

40. The cutting guide of claim 38, wherein the lower body portion includes retaining structure configured and dimensioned to inhibit relative movement between the donor tissue and the cutting guide.

41. The cutting guide of claim 40, wherein the retaining structure includes at least one opening configured and dimensioned to receive at least one fastener insertable into the donor tissue through the at least one opening.

42. A method of forming a tissue graft from donor tissue comprising:
positioning the donor tissue within a cutting guide;
inserting a cutting implement into a template of the cutting guide such that the cutting implement extends through the template into contact with the donor tissue; and
moving the cutting implement through a channel defined by the template to remove portions of the donor tissue and thereby form the tissue graft.

43. The method of claim 42, wherein positioning the donor tissue within the cutting guide includes positioning the donor tissue within an internal chamber collectively defined by upper and lower body portions of the cutting guide.

44. The method of claim 43, wherein positioning the donor tissue within the internal chamber includes separating the upper and lower body portions of the cutting guide.

45. The method of claim 42 further including removing the template from the cutting guide, inverting the template, and re-positioning the inverted template in the cutting guide.

46. The method of claim 45, wherein removing the template from the cutting guide includes removing a pair of arms extending outwardly from the template from cleats defined by the cutting guide.

47. The method of claim 46, wherein re-positioning the inverted template includes re-inserting the arms into the cleats.

48. The method of claim 45 further including moving the cutting implement through the channel of the template following inversion of the template to remove additional portions of the donor tissue.

49. The method of claim 42 further including securing the donor tissue in relation to the cutting guide.

50. The method of claim 49, wherein securing the donor tissue in relation to the cutting guide includes inserting at least one fastener into the donor tissue through at least one corresponding opening formed in the cutting guide.

51. A method of performing a surgical procedure comprising:
forming a tissue graft from donor tissue, wherein forming the tissue graft comprises:
positioning the donor tissue within a tissue graft cutting guide;
inserting a cutting implement into a template of the tissue graft cutting guide such that the cutting implement extends through the template into contact with the donor tissue; and
moving the cutting implement through a channel defined by the template to remove portions of the donor tissue and thereby form the tissue graft;
forming a recipient site within a patient's tissue, wherein forming the recipient site comprises:
deforming a recipient site cutting guide such that the recipient site cutting guide is contoured to the patient's tissue; positioning a cutting bit in engagement with the recipient site cutting guide; and
moving the cutting bit in relation to the recipient site cutting guide such that the cutting bit follows a path defined by a cutting route of the recipient site cutting guide to thereby remove portions of the patient's tissue and form the recipient site; and
positioning the tissue graft within the recipient site.

52. The method of claim 51, wherein deforming the recipient site cutting guide includes bending flexible material comprising a body of the recipient site cutting guide.

53. The method of claim 52, wherein deforming the recipient site cutting guide includes bending a body of the recipient site cutting guide at a weakened portion of the body.

54. The method of claim 53, wherein bending the body of the recipient site cutting guide includes bending the body at a relief formed in the body.

55. The method of claim 54 further including positioning the recipient site cutting guide in relation to the patient's tissue such that teeth defined by the relief contact the patient's tissue.

56. The method of claim 51 further including securing the recipient site cutting guide in relation to the patient's tissue.

57. The method of claim 56, wherein securing the recipient site cutting guide in relation to the patient's tissue includes inserting a mounting member into the patient's tissue through at least one opening formed in the recipient site cutting guide.

58. The method of claim 51, wherein positioning the cutting bit in engagement with the recipient site cutting guide includes positioning the cutting bit such that a cutting head of the cutting bit extends through a non-circular window of the recipient site cutting guide into contact with the patient's tissue.

59. The method of claim 58, wherein positioning the cutting bit includes positioning the cutting bit in engagement with a shoulder extending inwardly into the window of the recipient site cutting guide, the shoulder defining the cutting route.

60. The method of claim 59, wherein moving the cutting bit includes moving the cutting bit between discrete endpoints of the cutting route.

61. The method of claim 51, wherein positioning the donor tissue within the tissue graft cutting guide includes positioning the donor tissue within an internal chamber collectively defined by upper and lower body portions of the tissue graft cutting guide.

62. The method of claim 61, wherein positioning the donor tissue within the internal chamber includes separating the upper and lower body portions of the tissue graft cutting guide.

63. The method of claim 61 further including removing the template from the upper body portion, inverting the template, and re-positioning the inverted template.

64. The method of claim 63, wherein removing the template from the upper body portion includes removing a pair of arms extending outwardly from the template from cleats defined by the upper body portion.

65. The method of claim 64, wherein re-positioning the template includes re-inserting the arms into the cleats.

66. The method of claim 63 further including moving the cutting implement through the channel following inversion of the template to remove additional portions of the donor tissue.

67. The method of claim 51 further including securing the donor tissue in relation to the tissue graft cutting guide.

68. The method of claim 67, wherein securing the donor tissue in relation to the tissue graft cutting guide includes inserting at least one fastener into the donor tissue through at least one corresponding opening formed in the tissue graft cutting guide.

69. A method of forming a recipient site within a patient's tissue configured and dimensioned to receive a tissue graft, the method comprising:
deforming a recipient site cutting guide such that the recipient site cutting guide is contoured to the patient's tissue;
positioning a cutting bit in engagement with the recipient site cutting guide; and
moving the cutting bit in relation to the recipient site cutting guide such that the cutting bit follows a cutting route defined by the recipient site cutting guide to thereby remove portions of the patient's tissue and form the recipient site.

70. The method of claim 69, wherein deforming the recipient site cutting guide includes bending flexible material comprising a body of the recipient site cutting guide.

71. The method of claim 69, wherein deforming the recipient site cutting guide includes bending a body of the recipient site cutting guide at a weakened portion of the body.

72. The method of claim 71, wherein bending the body of the recipient site cutting guide includes bending the body at a relief formed in the body.

73. The method of claim 72 further including positioning the recipient site cutting guide in relation to the patient's tissue such that teeth defined by the relief contact the patient's tissue.

74. The method of claim 69 further including securing the recipient site cutting guide in relation to the patient's tissue.

75. The method of claim 74, wherein securing the recipient site cutting guide in relation to the patient's tissue includes inserting a mounting member into the patient's tissue through at least one opening formed in the recipient site cutting guide.

76. The method of claim 69, wherein positioning the cutting bit in engagement with the recipient site cutting guide includes positioning the cutting bit such that a cutting head of the cutting bit extends through a non-circular window of the recipient site cutting guide to facilitate contact with the patient's tissue.

77. The method of claim 76, wherein positioning the cutting bit includes positioning the cutting bit in engagement with a shoulder extending inwardly into the window of the recipient site cutting guide that defines the cutting route.

78. The method of claim 77, wherein positioning the cutting bit in engagement with the shoulder includes positioning a depth guide of the cutting bit in engagement with the shoulder.

79. The method of claim 78, wherein moving the cutting bit in relation to the recipient site cutting guide includes moving the depth guide along the shoulder.

80. The method of claim 69, wherein moving the cutting bit in relation to the recipient site cutting guide includes moving the cutting bit between discrete endpoints of the cutting route.
